(19) United States
(12) Patent Application Publication
(10) Pub. No.: US 2004/0062723 A1
(43) Pub. Date: Apr. 1, 2004

(54) DUAL COMPONENT TOOTH WHITENING DENTIFRICE

(76) Inventors: Lynette A. Zaidel, Cranford, NJ (US); Nora C. Lin, Basking Ridge, NJ (US)

Correspondence Address: COLGATE-PALMOLIVE COMPANY, 909 RIVER ROAD, PISCATAWAY, NJ, 08855, US

(21) Appl. No.: 10/065,244

(22) Filed: Sep. 27, 2002

(51) Int. Cl. A61K 7/20; A61K 9/16; A61K 9/50

(52) U.S. Cl. 424/53

(57) ABSTRACT

A peroxide containing dual component dentifrice composition providing enhanced whitening efficacy and minimal gingival irritation and tooth sensitivity, the composition being comprised of a separately maintained first aqueous dentifrice component containing a peroxide compound and an abrasive system compatible with the peroxide and a second aqueous dentifrice component containing a desensitizing compound and an abrasive that is incompatible with peroxide compound whereby when the components are mixed and combined for the first time and exposed to the surface of teeth, an enhanced whitening effect is obtained with minimal tooth sensitivity and gum irritation.

### BACKGROUND OF INVENTION

Field of the Invention

This invention relates generally to a composition for whitening human teeth, and more particularly, to a dual component composition which when the combined components are contacted with tooth surfaces more rapidly effects tooth whitening with minimal tooth sensitivity and gum irritation.

Many substances that a person confronts or comes in contact with on a daily basis can “stain” or reduce the “whiteness” of one's teeth. In particular, the foods, tobacco products and beverages such as tea and coffee that one consumes tend to stain one's teeth. These products tend to accumulate on the enamel layer of the tooth and form a pellicle film over the teeth. These staining and discoloring substances can then permeate the enamel layer. This problem occurs gradually over many years, but imparts a noticeable discoloration of tooth enamel. The majority of the population consider clean, white teeth to be aesthetically desirable. Dull-looking, stained teeth are objectionable to most people both on the basis of cosmetic appearance and also socially as an indication of poor oral hygiene.

Staining of teeth results from extrinsic and/or intrinsic staining. Extrinsic staining arises as a result of compounds such as tannins and other polyphenolic compounds that become trapped in and tightly bound to the proteinaceous layer on the surface of teeth. This type of staining can usually be removed by mechanical methods of tooth cleaning. In contrast, intrinsic staining occurs when staining compounds penetrate the enamel. This type of staining is not amenable to mechanical methods of tooth cleaning, and bleaching compounds which can penetrate into the tooth structure, are required. Intrinsic tooth staining is generally more intractable and difficult to remove than extrinsic tooth staining.

Among the strategies available for removing tooth stains, the most effective bleaching compositions contain a peroxy generating agent, such as hydrogen peroxide. Professional approaches to whitening a patient's teeth, include the construction of a dental bleaching tray made from an impression of the patient's dentition into which a peroxide is dispensed and worn daily for a period of from about 1 to 2 weeks, depending upon the severity of tooth staining. These peroxide compositions, usually packaged in a take home kit which is distributed by the dental professional which contains small plastic syringes or tubes which are dispensed directly by the patient into the tooth bleaching tray and held in place in the mouth for contact times ranging from 20 minutes to overnight. The most commonly used peroxide compositions contain 3-20% peroxide which is mixed with viscous vehicle and packaged in single barrel syringes. The dental professional who distributed the take home kit monitors the treatment during recall visits and remediates any tooth and gingival sensitivity issues.

Commonly assigned U.S. Pat. No. 5,171,564 discloses compositions for dentist supervised tooth whitening comprised of a gel vehicle to which is added a peroxide compound and a peroxide compatible abrasive such as calcium pyrophosphate or dicalcium phosphate. Commonly assigned U.S. Pat. No. 5,766,574 discloses a dual component whitening dentifrice composition providing enhanced whitening comprised of a first dentifrice component containing a peroxide compound and a second dentifrice component containing an abrasive, such as alumina or silica, which is incompatible with the peroxide, the first and second dentifrice components being maintained separate from the other until dispensed and combined for application to teeth requiring whitening.

Although the compositions disclosed in U.S. Pat. No. 5,171,564 and U.S. Pat. No. 5,766,574 are effective for tooth whitening, the whitening is accomplished at a relatively slow rate which inhibits full acceptance and use compliance of these compositions by consumers. Although more rapid whitening can be achieved by using higher peroxide concentrations, higher than those disclosed in such patents, that is greater than 10% by weight, patients using these compositions at these relatively high concentration levels often encounter tooth sensitivity and gingival irritation problems at the increased peroxide levels. Commonly assigned U.S. Pat. No. 6,180,089 discloses a dual component dentinal desensitizing dentifrice which although effective in ameliorating dentinal hypersensitivity has no effect on tooth whitening. Therefore, there is a need in the marketplace for a peroxide containing dentifrice which rapidly effects tooth whitening at peroxide concentrations which do not raise issues of tooth sensitization or gum irritation in the patient receiving the whitening treatment.

### SUMMARY OF INVENTION

The present invention encompasses a rapid acting dual component dental whitening composition based upon the discovery that when a first aqueous dentifrice component comprised of a separately maintained dentifrice component containing a peroxide tooth whitening agent and an abrasive system compatible with the peroxide whitening agent and an second aqueous dentifrice component containing a tooth desensitizing agent and an abrasive that is incompatible with peroxide compound, are mixed and combined for the first time and applied to the surface of stained teeth, an enhanced whitening effect is obtained without causing meaningful issues of tooth sensitivity or gum irritation.

In one embodiment of the invention, a dual component dental whitening composition is provided which is comprised of a first aqueous component containing a peroxide compound such as hydrogen peroxide, at relatively low concentration levels, and a calcium pyrophosphate abrasive compatible with the peroxide compound and a second aqueous dentifrice component containing a desensitizing agent such as a potassium salt and an abrasive such as silica or alumina which abrasives are incompatible with the peroxide compound. The individual components are maintained separate from each other and are not combined and admixed until simultaneous application to teeth is performed by the patient. Unexpectedly, when the separately maintained dentifrice components are combined in a suitable receptacle such as a dental tray immediately prior to application to teeth, rapid whitening of the teeth is obtained, which as will be subsequently demonstrated, is substantially more rapid than that achievable with the peroxide dentifrice component when used in the absence of the incompatible abrasive containing dentifrice component.

### DETAILED DESCRIPTION

Peroxide Whitening Dentifrice Component In the practice of the present invention the dentifrice component containing the peroxide ingredient is formulated as a paste using an aqueous vehicle containing a peroxide compound as the whitening agent, a thickening agent such as a polyoxyethylene/polyoxypropylene block copolymer and a peroxide compatible abrasive such as a calcium phosphate salt.

Peroxide Examples of suitable peroxide compounds used to prepare the dentifrice whitening component of the present invention include metal ion free peroxide ingredients such as hydrogen peroxide and organic peroxides such as urea peroxide, glyceryl peroxide and benzoyl peroxide as well as metal ion containing peroxides such as calcium peroxide and sodium percarbonate. A preferred peroxide compound is hydrogen peroxide.

Typically, the peroxide compound is employed in the composition of the present invention in amounts so that at least about 3% by weight of the whitening component comprises a peroxide compound up to about 30% by weight. Preferably, when the peroxide compound is hydrogen peroxide, the peroxide comprises from about 5 to about 10% by weight of the dentifrice whitening component.

Vehicle Humectants such as glycerin and polyethylene glycol in combination with water are useful in preparing the vehicle for the peroxide whitening component of the present invention. A combination of glycerine, polyethylene glycol and water is preferred as the vehicle in which the other ingredients of the peroxide component are contained.

Illustrative of polyethylene glycols useful in the practice of the present invention include nonionic polymers of ethylene oxide having the general formula:

HOCH₂ (CH₂OCH₂)ₙCH₂OH

Wherein n represents the average number of oxyethylene groups. These polyethylene glycols are designated by a number such as 400, 600, 800, 1000. which represents the average molecular weight of the glycol. The average molecular weight of the polyethylene glycols used in the practice of the present invention is about 200 to 1000, preferably 400 to 800 and most preferably 600 (PEG 600).

Glycerin and polyethylene glycol are included in the peroxide dentifrice component of the present invention in an amount of from about 2 to about 40% by weight and preferably about 5 to about 30% by weight. Water is incorporated in the aqueous whitening dentifrice component of the present invention at a concentration of about 20 to about 40 by weight of the composition and preferably about 25 to about 35% by weight.

Thickening Agent Thickening agents used in the preparation of the peroxide whitening dentifrice component include polyoxyethylene/polyoxypropylene block copolymers. Illustrative of polyoxyethylene/polyoxypropylene block copolymers useful in the practice of the present invention include block copolymers having the formula

HO(C₂H₄O)_{b}(C₃H₆O)ₐ(C₂H₄O)_{b}H

wherein a is an integer such that the hydrophobic portion (moiety) represented by (C3H6O) has a molecular weight of about 2750 to 4000, b is an integer such that the hydrophilic portion represented by (C2H4O) constitutes about 70-80% by weight of the copolymer. Block copolymers of this composition are available commercially under the trademark Pluronic F type. Pluronic F127, which has a molecular weight of 4000 and contains 70% of the hydrophilic C2H40 moiety is preferred in the practice of the present invention.

Another example of suitable thickening agents are nonionic poly (ethylene oxide) polymers available under the trademark Polyox having molecular weights in the range of 100,000 to 1,000,000. Polyox PEG 2M available from Union Carbide having a molecular weight of about 100,000 is preferred for use as a thickening agent in the practice of the present invention.

The thickening agent is preferably present in the peroxide dentifrice component in an amount within the range of about 5 to about 30% by weight and about 15 to about 25% by weight is preferred.

Calcium Phosphate Abrasives Calcium phosphate based abrasives which have been found to be compatible with peroxide compounds are incorporated in the peroxide dentifrice component in accordance with the teachings of U.S. 5,171,564, which teachings are incorporated herein by reference. Examples of such peroxide compatible calcium phosphate abrasives include dicalcium phosphate dihydrate and anhydrous dicalcium phosphate or calcium pyrophosphate.

The calcium phosphate abrasive is advantageously incorporated in the peroxide dentifrice component at a concentration of about 20 to about 60% by weight and preferably about 20 to about 30% by weight. These abrasive concentrations are believed to serve to increase the density of the peroxide dentifrice component and concentrate the peroxide for better whitening efficacy, as will hereinafter be demonstrated. When formulated in accordance with the practice of the present invention, the peroxide component has pH in the range of about 4.0 to about 7.0 and preferably about 5.5 to about 6.8.

Chelating Agents Chleating agents to provide peroxide storage stability may be used to prepare the peroxide whitening dentifrice component and such agents include sodium acid pyrophosphate, disodium calcium ethylene diamine tetraacetic acid (Na2Ca EDTA), phosphoric acid, citric acid, sodium citrate, potassium citrate, sodium phosphate, sodium pyrophosphate, potassium pyrophosphate, potassium stannate and disodium ethylenediamine tetraacetate. The presence of the chelating agent will inactivate any metal impurities which act to destabilize the peroxide ingredient. The chelating agent if present in the peroxide whitening dentifrice component is incorporated in the dentifrice component in an amount within the range of 0.1 to about 8.0% by weight and preferably about 0.5 to about 3.0% by weight. Chelating agents may also be introduced by use of commercially available 35% hydrogen peroxide stock reagents that are manufactured with chelating stabilizers such as potassium stannate, sodium phosphate or phosphoric acid.

Peroxide Whitening Dentifrice Component Preparation The peroxide whitening component is prepared in the form of an aqueous paste. Preparation is carried out at room temperature. The peroxide, sweeteners, chelating agents, and water are premixed until all solids dissolve. The humectants and thickening agents are mixed in a Ross mixer under 24-28 Hg vacuum until a uniform mixture results. The aqueous mixture is then added and the resulting mixture is stirred at high speed until a clear gel forms. The calcium phosphate abrasives and flavor are added and the mixture stirred until uniformly dispersed.

Second Desensitizing Dentifrice Component The aqueous dentifrice component in which a desensitizing agent and peroxide incompatible abrasive material is included is generally prepared as a paste using a vehicle which contains water, humectant, surfactant and thickener.

Vehicle The humectant used in the preparation of the second aqueous dentifrice component containing a desensitizing agent and a peroxide incompatible abrasive is generally a mixture of humectants, such as glycerol, and a polyethylene glycol of molecular weight in the range of 200 to 1000, but other mixtures of humectants and single humectants may also be employed. The humectant content in the aqueous dentifrice component is in the range about of 20% to about 60% by weight and preferably about 20 to about 40% by weight of the dentifrice component. The water content is in the range of about 20 to about 40% by weight and preferably about 25 to about 35% by weight.

Desensitizing Agent A source of potassium ion is incorporated in the second dentifrice component to provide desensitizing properties. The potassium ion is sourced generally from water soluble potassium salt including potassium nitrate, potassium citrate, potassium chloride, potassium bicarbonate and potassium oxalate with potassium nitrate being preferred. The potassium salt is generally incorporated in one or more of the dentifrice components at a concentration of about 0.5 to about 20% by weight and preferably about 3 to about 15% by weight.

Thickening Agent Thickeners include organic and inorganic thickeners. Inorganic thickeners which may be included in the dentifrice components include amorphous silicas such as Zeodent 165 available from Huber Corporation, and Sylox 15 from W. R. Grace.

Organic thickeners of natural and synthetic gums and colloids may also be used to prepare the dentifrice components of the present invention. Examples of such thickeners are carrageenan (Irish moss), xanthan gum, sodium carboxymethyl cellulose, starch, polyvinylpyrrolidone, hydroxyethylpropyl cellulose, hydroxybutyl methyl cellulose, hydroxypropyl methyl cellulose, and hydroxyethyl cellulose as well as nonionic thickening agents such as Pluronic F127.

The inorganic thickener may be incorporated in the dentifrice components of the present invention at a concentration of about 1.0 to about 5% by weight and preferably about 2 to about 4% by weight. The organic thickener may be incorporated in the compositions of the present invention at a concentration of about 0.5 to about 5% by weight and preferably about 2 to about 4% by weight.

Abrasives Abrasives incompatible with peroxide compounds which may be incorporated in the desensitizing second dentifrice component of the present invention include siliceous materials, such as silica. A preferred silica is a precipitated amorphous hydrated silica, such as Sorbosil AC-35, marketed by Crosfield Chemicals, or Zeodent 115 from Huber Company but other abrasives may also be employed, including hydroxyapatite, sodium metaphosphate, potassium metaphosphate, calcium carbonate, sodium bicarbonate, alumina trihydrate, aluminum silicate and calcined alumina.

The concentration of abrasive in the desensitizing second dentifrice component of the present invention will normally be in the range of 2 to about 40% by weight and preferably 5 to 25% by weight.

Alkaline agents such as alkali metal compounds including sodium hydroxide, potassium hydroxide, sodium bicarbonate and sodium carbonate may be incorporated in the second dentifrice component of the present invention in amounts in the range of about 0.5 to 15% by weight, preferably about 1.0 to about 8% by weight and most preferably at about 1.0 to about 5.0% by weight of the component. Mixtures of the above alkali metal compounds may also be used. Sodium hydroxide is the preferred alkaline agent. The pH of the second component is in the range of about 8 to about 13. The pH of the mixture of the dual components of the present invention ranges from about 8 to about 11.

Surfactants Surfactants may be incorporated in the second dentifrice component to provide foaming properties to the combined dentifrice components. The surfactant is preferably anionic in nature. Suitable examples of anionic surfactants are higher alkyl sulfates such as potassium or sodium lauryl sulfate which is preferred, higher fatty acid monoglyceride monosulfates, such as the salt of the monosulfated monoglyceride of hydrogenated coconut oil fatty acids, alkyl aryl sulfonates such as sodium dodecyl benzene sulfonate, higher fatty sulfoacetates, higher fatty acid esters of 1,2 dihydroxy propane sulfonate.

The surfactant is generally present in the second desensitizing dentifrice component of the present invention at a concentration of about 0.2 to about 5% by weight and preferably about 0.3 to about 1% by weight.

Colorants Colorants such as pigments and dyes may be used in the practice of the present invention. Pigments include nontoxic, water insoluble inorganic pigments such as titanium dioxide and chromium oxide greens, ultramarine blues and pinks and ferric oxides. The pigments have a particle size in the range of 5-1000 microns, preferably 250-500 microns, and are present at a concentration of 0.5 to 3% by weight.

Dyes used in the practice of the present invention are generally food color additives presently certified under the Food Drug & Cosmetic Act for use in the food and ingested drugs, including dyes such as FD&C Red No. 3 (sodium salt of tetraiodofluorescein), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulffobenzylamino)-phenyl]-(4-hydroxy-2-sulfoniumphenyl)-mewthylene}-[1-(N-ethyl-N-p-sulfobenzyl)-Δ-3,5-cyclohexadienimine], FD&C Blue No. 1 (disodium salt of dibenzyldiethyldiaminotriphenylcarbinol trisulfonic acid of indigotin) and mixtures thereof in various proportions. The concentration of the dye for the most effective result in the present invention is present in the dentifrice composition in an amount from about 0.0005 percent to about 1 percent of the total weight.

The incorporation in the dual component composition of the present invention of peroxide sensitive dyes which react with and gradually lose their color on contact with the peroxide ingredient can act as a signal to alert the patient that the whitening treatment is completed and the mouth tray or other receptacle and its dual component contents may be removed from the users teeth. To effect such color signal, a dye which is reactive with peroxide compounds is incorporated in the second dentifrice component containing the desensitizing agent and peroxide incompatible abrasive. The individual components when initially combined are colored by the presence of the dye which slowly fades over the treatment period. FD&C blue #1, FD&C blue #2, FD&C Green #3, Yellow #6, and FD&C red #40 are peroxide sensitive dyes which fade upon exposure to peroxide. Of these, FD&C Green #3 and Yellow #6 were the most useful color signals (blue and orange, respectively at alkaline pH), having good stability in the second desensitizing component dentifrice, and gradually losing color upon exposure to peroxide component over a desired treatment time of 10 to about 30 minutes.

The abrasives present in the mixture of dual dentifrice components of the present invention impart an added benefit by providing an opaque background, making the dye more visible when the composition is applied to the teeth than if the dye were added to a transparent, nonabrasive gel. This aids the patient during product application, ensuring uniform coverage of product on all areas of the teeth.

Flavorings and Sweeteners Any suitable flavoring or sweetening material may also be incorporated in the second dentifrice component of the present invention. Examples of suitable flavoring constituents include flavoring oils, as for example, oils of spearmint, peppermint, wintergreen, sassafras, clove, sage, eucalyptus, marjoram, cinnamon lemon, and orange, and methyl salicylate. Suitable sweetening agents include sucrose, lactose, maltose, sorbitol, xylitol, sodium cyclamate, perillatine, and sodium saccharin. Suitably, flavor and sweetening agents may together comprise from 0.01% to 5% or more of the dentifrice components.

Second Desensitizing Component Preparation To prepare the second desensitizing dentifrice component of the present invention, generally the humectants such as glycerin and polyethylene glycol are dispersed along with any organic thickeners. Next, a premixed solution of warm water (about 50° C.), sweeteners, desensitizing agents, dyes, and an alkaline agent such as sodium hydroxide and/or sodium bicarbonate/carbonate is added. These ingredients are mixed until a homogenous phase is obtained for each component. Thereafter inorganic thickener, a peroxide incompatible abrasive such as a silica abrasive, flavor and surfactant ingredients are added and the ingredients mixed at high speed under vacuum of 24-28 of Hg. The resultant product is a homogeneous, semiextrudible paste product.

Dentifrice Dispensers The multicomponent dentifrice composition of the present invention is packaged in a suitable dispensing container in which the components are maintained physically separated and from which the separated components may be dispensed substantially synchronously as a combined ribbon for application to receptacle such as a dental tray. Such dispensers are known in the art. Advantageously each component is kept isolated in a separate compartment of the dispenser. Advantageously, the dispenser will simultaneously deliver approximately equal weight amounts of each component through an orifice at whose end the separate components may intermingle. An example of a dispenser suitable for use in the practice of the present invention includes a two chamber dispensing container, such as a pump or a tube, as disclosed in U.S. Pat. Nos. 4,487,757 and 4,687,663; wherein, the tube body is formed from a collapsible plastic web such as polyethylene or polypropylene and is provided with a partition within the container body defining separate compartments in which the physically separated components are stored and from which they are dispensed through a suitable dispensing outlet.

A preferred means for dispensing the dual component composition of the present invention is the use of a double barreled syringe adapted for mixing and dispensing two component viscous materials. The chambers in the syringe are filled with the appropriate amount of the dentifrice components. Such double barreled syringes are equipped with a plunger for dispensing equal weight amounts of the components of the two component dentifrice which form an admixture when the individual components are simultaneously discharged into a mixing tip having a static mixing element. Thereafter, the combined components are discharged into a dental tray or similar receptacle for use in whitening teeth. Double barreled syringes useful for dispensing the dual component whitening composition of the present invention are available commercially from Plas-Pak Industries, Inc., Norwich, Conn.

The following example is further illustrative of the present invention, but it is understood that the invention is not limited thereto. All amounts and proportions referred to herein and in the appended claims are by weight, unless otherwise stated.

Example A two component tooth whitening dentifrice of the present invention was prepared, designated Composition X, which was comprised of Component A, a peroxide containing tooth whitening paste and Component B, a desensitizing paste. The ingredients of Components A and B are listed in Table I below.

TABLE I
Composition X

| | A Peroxide | B Desensitizing |
|---|---|---|
| Component | Paste | Paste |
| Ingredients | Weight % | Weight % |
| Deionized Water | 24.00 | 30.2 |
| Hydrogen peroxide | 10.00 | — |
| Potassium Nitrate | — | 10.00 |
| Polyox (PEG 2M) | 5.00 | — |
| Glycerin | 5.0 | 25.0 |
| Polyethylene glycol 600 | 10.00 | 3.000 |
| Xanthan | — | 0.700 |
| Na carboxymethyl cellulose | — | 0.500 |
| Pluronic F-127 | 20.0 | 2.000 |
| Sodium saccharin | — | 0.200 |
| Titanium dioxide | — | 2.000 |
| Sodium hydroxide (50%) | — | 1.000 |
| TiO₂ | — | 1.00 |
| FD + C Green #3 | — | ` 0.025 |
| Zeodent 115 (silica abrasive) | — | 17.5 |
| Zeodent 165 (silica thickener) | — | 3.000 |
| Sodium bicarbonate | — | 5.000 |
| Calcium pyrophosphate | 25.00 | — |
| Sodium lauryl sulfate | — | 0.400 |
| Flavor | 0.5 | 0.50 |

Components A and B were prepared in a Ross mixer as follows: Component A Water, saccharin and 30% of the total volume of 35% hydrogen peroxide were stirred in a beaker until the saccharin dissolved. PEG 600 and glycerin were added and the mixture was placed in the Ross mixer along with Pluronic F127 and stirred at high speed under vacuum until a clear gel formed (approximately 45 minutes). Calcium pyrophosphate was added and the mixture stirred at medium speed for another 10 minutes under vacuum. The remaining 70% of the peroxide volume was added and stirred 10 minutes at medium speed. Next, the flavor was added and the mixture was stirred 5 minutes at low speed. An extrudible paste having a pH of 6.0 was obtained.

Component B Water, sodium hydroxide, potassium nitrate and FD+C Green #3 dye were mixed in a beaker until the potassium nitrate dissolved. PEG 600 and glycerin were added to the aqueous phase and the mixture was placed in the Ross mixing pot along with Pluronic F127. The mixture was stirred at high speed under vacuum until a clear gel formed (approximately 45 minutes). Calcium pyrophosphate, sodium lauryl sulfate, sodium bicarbonate and sodium hydroxide were added and the mixture stirred at medium speed for another 10 minutes under vacuum. An extrudable paste having a pH of 9.6 was obtained.

Whitening Efficacy Studies Study I—In Vitro AssessmentStain removal by Composition X was determined by an in vitro study procedure using human extracted teeth that were polished with a prophy paste to remove any surface stain. The root portions of five teeth were placed in a row and immersed in an impression compound. To mimic in vivo use, a tray for the sample teeth was prepared by placing a piece of tray material in a tray former and heated until the material began to soften. The softened tray material was then pulled down over the five teeth using vacuum and allowed to harden to entrap the teeth in the hardened tray material.

Baseline Chromameter readings were taken of the teeth using a Minolta Chromameter CR-241. Next, small dots of Composition X composed of equal weight amounts of Component A and B, so that the peroxide content was 5% by weight, were placed in each tooth well in a dental tray. The tray was weighed and then placed over the teeth so that a thin film of Composition X covered each tooth. The tray covered teeth were placed in a 37° C. incubator for 15 minute intervals and thereafter removed, rinsed and measured with the Chromameter. This process was repeated for each time point. The Δ E values were measured for each time point. The increase in tooth whiteness (Δ E) was calculated using the following formula:

ΔE=[ΔL)²+(Δa)²+(Δb)²]^{1/2}

wherein the higher the value of A E, the higher is the level of achieved tooth whiteness.

For purposes of comparison, the single component peroxide paste Component A alone (designated Composition C) was evaluated for tooth whitening efficacy following the procedure of the Example except that ΔE values of the tray covered teeth were measured at 30 minute intervals. For purposes of further comparison, a commercially available professional tooth whitening composition was also evaluated for tooth whitening efficacy with chromameter measurements being performed also at 30 minute intervals The commercial whitening composition, designated “Composition C1”, was a thickened Pluronic gel which contained 7.5% peroxide at pH 9. No abrasives were present in the gel. The whitening study was carried out to 14 treatments, which is equivalent to a typical home whitening regimen of twice daily treatments over a one week period. The results are recorded in Table II.

TABLE II
Tooth Whitening Efficacy

| | |
|---|---|
| No. of | |
| Treatments | Delta E |

| Composition | 4 | 8 | 12 | 14 |
|---|---|---|---|---|
| X | 6.98 | 7.82 | 8.07 | 8.23 |
| C | 2.71 | 4.26 | 5.55 | 5.84 |
| C1 | 4.47 | 7.12 | 8.16 | 8.38 |

The results recorded in Table II indicate that although the treatment time (15 minutes) using Composition X was half the treatment time (30 minutes) of Composition C1, Composition X achieved faster maximum whitening in half the number of treatments and half the application time inspite of the fact that Composition C1 contained 33% more peroxide whitening agent than Composition X (7.5% v. 5.0%).

This unexpected enhancement in Composition X of whitening efficacy over Composition C1 is believed to be due to the presence in the composition of calcium pyrophosphate and silica abrasives which serve to aid in extrinsic stain removal and, boost the effective peroxide concentration delivered inside the tooth by the increased density and solid content of the Composition X formula as compared to Composition C1 Thus, larger amounts of peroxide were delivered by Composition X to the teeth per unit weight of paste. In addition, the peroxide ingredient in Composition X was concentrated into the water soluble portion of the composition and it is this concentrated portion which is believed to have diffused into the teeth to remove intrinsic stain.

It is further noted that the dual component Composition X when compared to the single component Composition C also provided significantly faster, greater whitening efficacy than Composition C although Composition C had the same peroxide content as Composition X and the same calcium pyrophosphate abrasive content.

Study II—In Vivo Assessment The whitening efficacy of Composition X composed of equal weight amounts of Components A and B was also compared to Composition C1 in a two week human clinical study wherein one cell of 9 patients used Composition C1 thirty minutes twice daily according to the manufacturer's instruction. The other cell with 12 patients used Composition X for fifteen minutes once daily. Tooth shade was evaluated after 0, 5, 7 and 14 days using a value-ordered Vita shade guide. The results are summarized in Table III.

TABLE III
Shade Guide Change

| | | Average | | | Average Shade | | |
|---|---|---|---|---|---|---|---|
| | Average | Shade Guide | | | Guide Change | | |
| | Shade at | After Day | | | After Day | | |
| Composition | Baseline | 5 | 7 | 14 | 5 | 7 | 14 |
| X | D3 | 7.67 | 6.15 | 4.88 | 3.58 | 4.92 | 6.19 |
| C1 | D3 | 8.06 | 6.11 | 4.22 | 2.28 | 3.92 | 5.66 |

The results recorded in Table III indicate that the overall whitening efficacy of Composition X was directionally better than Composition C1 and statistically better at 5 days (p=0.025) evidencing faster whitening efficacy since both the application time and number of treatments using Composition X was one quarter that of Composition C1 (15 minutes once daily versus 30 minutes twice daily).

Tooth Sensitivity and Gum Irritation Study In addition to enhanced efficacy, the patients in the tooth whitening study using Composition X as described above reported less gingival irritation and tooth sensitivity than patients using Composition C1. The patients involved in the study rated their tooth sensitivity and gingival irritation on a scale of zero (none) to 5, the higher the number, the greater the tooth sensitivity and gingival irritation experienced by the patient involved in the study. Patient rated their perception of tooth sensitivity and gingival irritation using this scale before using the product (baseline) and after using the product for 7 days and 14 days. The patient ratings of gum irritation and tooth sensitivity minus the baseline ratings are recorded in Table IV below.

TABLE IV
Change in tooth sensitivity and gum irritation from baseline

| | |
|---|---|
| Gum Irritation | Tooth Sensitivity |
| Day | Day |

| Composition | 7 | 14 | 7 | 14 |
|---|---|---|---|---|
| X | −0.08 | 0.00 | −0.62 | −0.46 |
| C1 | +1.11 | +1.13 | 0.00 | +0.33 |

Ratings for gum irritation and tooth sensitivity were lower for composition X compared to composition C1 at 7 and 14 days. The levels of tooth sensitivity reported for composition X at 7 and 14 days were negative (lower than the reported baseline values), suggesting that the desensitizing agent present in the composition reduced tooth sensitivity over the treatment period.

What is claimed is:

1. A dual component dentifrice composition providing enhanced whitening efficacy and minimal gingival irritation and tooth sensitivity, the composition being comprised of a separately maintained first aqueous dentifrice component containing a peroxide compound and an abrasive system compatible with the peroxide and a second aqueous dentifrice component containing a desensitizing compound and an abrasive incompatible with the peroxide compound whereby the components when mixed and combined for the first time and exposed to the surface of teeth, an enhanced whitening effect is rapidly obtained in the substantial absence of tooth sensitivity and gum irritation.

2. The composition of claim 1 wherein the peroxide compound is hydrogen peroxide.

3. The composition of claim 1 wherein the peroxide compound is urea peroxide.

4. The composition of claim 1 wherein the peroxide compatible abrasive system is comprised on one or more calcium phosphate salts.

5. The composition of claim 4 wherein the calcium phosphate salt is calcium pyrophosphate.

6. The composition of claim 1 wherein the desensitizing compound is a potassium salt.

7. The composition of claim 6 wherein the potassium salt is potassium nitrate.

8. The composition of claim 1 wherein the pH of the second component dentifrice is adjusted with an alkali metal hydroxide or carbonate salt.

9. The composition of claim 1 wherein the concentration of peroxide compound present in the combined dentifrice components ranges from 3 to about 30% by weight.

10. The composition of claim 1 wherein the concentration of desensitizing agent in the combined dentifrice components ranges from 3 to 10% by weight.

11. The composition of claim 1 wherein a peroxide reactive dye is present in the second dentifrice component, whereby reaction of the peroxide in the first component with the dye present in the second component when the components are combined for use in tooth whitening causes the original dye color to fade, signaling to the user the exhaustion of the peroxide content and completion of the whitening treatment.

12. The composition of claim 11 wherein the peroxide reactive dye is FD&C Green #3.

13. The composition of claim 1 wherein the pH of the first peroxide component is in the range of about 4 to about 7 and the second component has a pH in the range of about 8 to about 13.

14. The method for providing enhanced whitening efficacy and minimal gingival irritation and tooth sensitivity, the method comprising preparing a dual component composition comprised of a separately maintained first aqueous dentifrice component containing a peroxide compound and an abrasive system compatible with the peroxide and a second aqueous dentifrice component containing a desensitizing compound and an abrasive incompatible with the peroxide compound, mixing the components and contacting the mixed components with the surface of teeth whereby an enhanced whitening effect is rapidly obtained in the substantial absence of tooth sensitivity and gum irritation.

15. The method of claim 14 wherein the peroxide compound is hydrogen peroxide.

16. The method of claim 14 wherein the peroxide compound is urea peroxide.

17. The method of claim 14 wherein the abrasive system compatible with the peroxide is comprised of one or more calcium phosphate salts.

18. The method of claim 17 wherein the calcium phosphate salt is calcium pyrophosphate.

19. The method of claim 14 wherein the desensitizing compound is a potassium salt.

20. The method of claim 19 wherein the potassium salt is potassium nitrate.

21. The method of claim 14 wherein the pH of the second component dentifrice is adjusted with an alkali metal hydroxide or carbonate salt.

22. The method of claim 14 wherein the concentration of peroxide compound present in the combined dentifrice components ranges from 3 to about 30% by weight.

23. The method of claim 14 wherein the concentration of desensitizing agent in the combined dentifrice components ranges from 3 to 10% by weight.

24. The method of claim 14 wherein a peroxide reactive dye is present in the second dentifrice component, whereby reaction upon mixing of the peroxide in the first component with the dye in the second component when the components are combined for tooth whitening causes the dye color to fade signaling the exhaustion of the peroxide content and completion of the whitening treatment.

25. The method of claim 24 wherein the reactive dye is FD&C Green #3.

26. The method of claim 14 wherein the first peroxide component has a pH in the range of about 4 to about 7, the second component has an pH in the range of about 8 to about 13.
